SEQUENCE LISTING (16 sequences)

SEQ ID NO: 1 - protein, 12 aa, Homo sapiens
THRPPMWSPVWP

SEQ ID NO: 2 - protein, 11 aa, Homo sapiens
MQLPLATGGGC

SEQ ID NO: 3 - protein, 12 aa, Homo sapiens
FCDGFYACYMDV

SEQ ID NO: 4 - protein, 12 aa, Homo sapiens
YHWYGYTPQNVI

SEQ ID NO: 5 - protein, 10 aa, Homo sapiens
CTTHWGFTLC

SEQ ID NO: 6 - protein, 20 aa, Homo sapiens
RGDLATLRQLAQEDGVVGVR

SEQ ID NO: 7 - protein, 12 aa, Homo sapiens
GSETWKTIITKN

SEQ ID NO: 8 - protein, 7 aa, Homo sapiens
RKRLDRN

SEQ ID NO: 9 - protein, 9 aa, Homo sapiens
CVKTPAQSC

SEQ ID NO: 10 - protein, 9 aa, Homo sapiens
CDCRGDCFC

SEQ ID NO: 11 - protein, 4 aa, Homo sapiens
RGDS

SEQ ID NO: 12 - protein, 7 aa, Homo sapiens
NSVRGSR

SEQ ID NO: 13 - protein, 10 aa, Homo sapiens
EHWSYGLRPG

SEQ ID NO: 14 - protein, 19 aa, Homo sapiens
TFFYGGSRGKRNNFKTEEY

SEQ ID NO: 15 - protein, 9 aa, Homo sapiens
KRTGQYKLC

SEQ ID NO: 16 - protein, 12 aa, Homo sapiens
YHWYGYTPQNVI

(19) United States
(12) Patent Application Publication
(10) Pub. No.: US 2025/0064972 A1
(43) Pub. Date: Feb. 27, 2025

(54) METHOD FOR TREATING CANCER

(71) Applicant: Suntec Medical, Inc., Walnut, CA (US)

(72) Inventors: Chun-Ting Cheng, Taipei (TW); Yuan-Chung Tsai, Taipei (TW); Pauline Ying Lau, Walnut, CA (US)

(21) Appl. No.: 18/941,666

(22) Filed: Nov. 8, 2024

Related U.S. Application Data
(63) Continuation of application No. PCT/US2023/022744, filed on May 18, 2023.
(60) Provisional application No. 63/343,167, filed on May 18, 2022.

(51) Int. Cl. A61K 47/69; A61K 9/51; A61P 35/00; B82Y 5/00

(52) U.S. Cl. CPC A61K 47/6935; A61K 9/5146; A61K 9/5169; A61P 35/00; B82Y 5/00

(57) ABSTRACT

The present invention provides a conjugate comprising: (a) a cancer-targeting ligand, (b) a hydrophilic polymer of polyethylene glycol (PEG), polylactic acid (PLA), polylactic-co-glycolic acid (PLGA), or dextran, and (c) a flavonoid. The present invention also provides to a micelle nanoparticle composition comprising: (a) an outer shell comprising the conjugate, (b) an inner shell comprising oligomeric (−)-epigallocatechin gallate (OEGCG), and optionally (c) a cancer-treating molecule encapsulated in the inner shell. In one embodiment, the nanoparticle composition has at least 70% of the nanoparticles with a diameter between 20-500 nm or 50-300 nm, and one single major peak in the size distribution. The present invention further provides a method for treating cancer by administering an effective amount of the present nanoparticle composition to a subject. The cancer-targeting ligand targets the tumor and delivers active ingredients to tumor for treating cancer.

### CROSS-REFERENCE TO RELATED APPLICATION(S)

This application is a continuation of PCT/US2023/022744, filed May 18, 2023; which claims the benefit of U.S. Provisional Application No. 63/343,167, filed May 18, 2022. The contents of the above-identified applications are incorporated herein by reference in their entireties.

### SEQUENCE LISTING

This application contains an ST.26 compliant Sequence Listing, which was submitted in xml format via Patent Center and is hereby incorporated by reference in its entirety. The .xml copy, created on May 17, 2023 is named 140695.8007.WO01 Sequence List and is 14400 bytes in size.

### FIELD OF THE INVENTION

The present invention relates to a conjugate comprising: (a) a cancer-targeting ligand, (b) a hydrophilic polymer of polyethylene glycol (PEG), polylactic acid (PLA), polylactic-co-glycolic acid (PLGA), or dextran, and (c) a flavonoid, wherein the hydrophilic polymer covalently binds to the flavonoid and the cancer-targeting ligand. The present invention relates to micelle nanoparticles comprising: (a) an inner shell comprising oligomeric (−)-epigallocatechin gallate (OEGCG), (b) an outer shell comprising a cancer-targeting ligand-hydrophilic polymer-EGCG conjugate, and optionally (c) a cancer treating agent encapsulated in the inner shell.

### BACKGROUND OF THE INVENTION

Green tea catechins have health benefits of prevention of cancers. Among tea catechins, (−)-epigallalocatechin-3-gallate (EGCG) is the most abundant and it plays a major role in the beneficial effects of green tea. EGCG possesses antioxidant, anti-inflammatory, and immune modulation effects. EGCG has also been shown to effectively inhibit tumor growth and metastasis by targeting multiple signal transduction pathways essential for cancer cell survival.

Despite these desirable activities, clinical applications of EGCG have been limited by its poor stability and low oral bioavailability. For instance, EGCG is unstable and easily decomposed under physiological environment. As a result, plasma concentrations of EGCG required to achieve a desired therapeutic effect cannot be reached following oral administration.

There are three major challenges to the treatment of cancer, a complicated disease with multiple signaling pathways. First, cancers are erupted from a person's immune dysfunctions. Immune modulation to restore host immune function is critical for long term treatment resolution. Second, a single therapeutic agent can only modify one disease pathway and has limited efficacy, drug-resistance, and non-response. Cancer cells can escape from a single agent treatment through alternative signaling pathways. Third, drug toxicity and non-effective dosing to target tissue are common challenges to cancer therapies because tumor size is a small fraction of body size. Only a small fraction of the drug administered reaches targeted tissue, and a majority of the drug enters non-targeted normal tissues which causes low efficacy to targeted tissue and high toxicity to normal tissues.

There is a need for a pharmaceutical composition and a drug delivery system that overcomes the challenges described above and effectively enters target tissue without potential toxicity.

### BRIEF DESCRIPTION OF THE DRAWINGS

FIG. 1 shows a micelle composition of the present invention, in which a drug molecule is encapsulated within the micelle, and the micelle comprises Ligand-PEG-EGCG conjugate in an outer shell and oligomeric EGCG (OEGCG) in an inner shell.

FIG. 2 shows a micelle composition of the present invention, in which a drug molecule is encapsulated within the micelle, and the micelle comprises Ligand-PEG-EGCG conjugate plus bare PEG-EGCG in an outer shell and oligomeric EGCG (OEGCG) in an inner shell.

FIG. 3 shows the chemical synthesis scheme of RGD-PEG-EGCG via conjugating the N-terminal of RGD peptide to HOOC-PEG-EGCG.

FIG. 4 shows the chemical synthesis scheme of TP12-PEG-EGCG via conjugating the N-terminal of TP12 peptide to HOOC-PEG-EGCG.

FIG. 5 shows the successful formulation of RGD-MINC-doxorubicin (A) and TP12-MINC-doxorubicin micelle (B).

FIG. 6 shows the tumor cell uptake of MINC-doxorubicin, RGD-doxorubicin or TP12(TfR)-MINC-doxorubicin by measuring fluorescence signals.

FIG. 7 shows the tumor cell viability under MINC-doxorubicin, RGD-MINC-doxorubicin, or TP12(TfR)-MINC-doxorubicin treatment.

FIG. 8 shows the chemical synthesis scheme of RGD-PEG-EGCG via conjugating the C terminal of RGD peptide to HO-PEG-EGCG.

FIG. 9 shows the chemical synthesis scheme of TP12-PEG-EGCG via conjugating the C terminal of TP12 peptide to HO-PEG-EGCG.

FIG. 10 shows the chemical synthesis scheme of Folate-PEG-EGCG via conjugating the COOH group of Folic acid to HO-PEG-EGCG.

FIG. 11 shows the chemical synthesis scheme of RGD-PLA-EGCG via conjugating the N-terminal of RGD peptide to HOOC-PLA-EGCG.

FIG. 12 shows the chemical synthesis scheme of RGD-PLGA-EGCG via conjugating the N-terminal of RGD peptide to HOOC-PLGA-EGCG.

FIG. 13 shows the chemical synthesis scheme of RGD-Dextran-EGCG via conjugating the C-terminal of RGD peptide to HO-Dextran-EGCG.

### DETAILED DESCRIPTION OF THE INVENTION

### Definitions

The term “about” is defined as +10%, preferably +5%, of the recited value.

The term “a cancer-targeting ligand”, as used herein, refers to a molecule of molecular weight <10000 Daltons, for example, 300-3500 Daltons, such as a peptide, an acid, or a carbohydrate, that binds or targets receptors on a cancer cell surface or tumor environment.

The term “cytokines” refer to proteins (˜5-70 kDa) important in cell signaling. Cytokines have been shown to be involved in autocrine, paracrine, and endocrine signaling as immunomodulating agents. Cytokines include interferons, interleukins, lymphokines, tumor necrosis factors, and chemokines.

The term “epigallocatechin gallate” refers to an ester of epigallocatechin and gallic acid, and is used interchangeably with “epigallocatechin-3-gallate” or EGCG.

The term “oligomeric EGCG” (OEGCG) refers to 3-20 monomers of EGCG that are covalently linked. OEGCG preferably contains 4 to 12 monomers of EGCG.

The term “nanoparticles” refers to particles with a diameter below 1p m and between 1-999 nm.

The term “polyethylene glycol-epigallocatechin gallate conjugate” or “PEG-EGCG refers to polyethylene glycol (PEG) conjugated to one or two molecules of EGCG. The term “PEG-EGCG” refer to both PEG-mEGCG conjugate (monomeric EGCG) and PEG-dEGCG (dimeric EGCG) conjugate.

The term “MINC” (Multi-pathway Immune-modulating Nanocomplex Combination therapy) is a platform technology. As used in this application, MINC utilizes the bioactivity of PEG-flavonoid conjugate and oligomeric EGCG (OEGCG). MINC can encapsulate additional cancer treating agents to form MINC-agent.

The term “MINC-agent”, as used in this application, is a micelle with a shell formed by cancer-targeting ligand-PEG-flavonoid conjugate and optionally oligomeric flavonoid such as OEGCG and has an agent encapsulated within the shell.

### Flavonoids

Flavonoids suitable for the present invention have the general structure of Formula I:

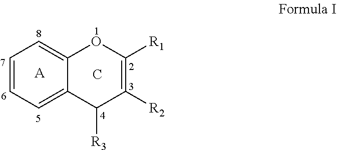

wherein:
R₁ is H, or phenyl;
R₂ is H, OH, Gallate, or phenyl; wherein the phenyl is optionally substituted by one or more (e.g., 2-3) hydroxyl;
R₃ is H, OH, or =O (oxo); or
R₁ and R₂ together form a close-looped ring structure; or
R₂ and R₃ together form close-looped ring structure.

The 2, 3, 4, 5, 6, 7, or 8 position of Formula I, can be linked to a group containing hydrocarbon, halogen, oxygen, nitrogen, sulfur, phosphorus, boron or metals.

Examples of flavonoids of Formula I include:

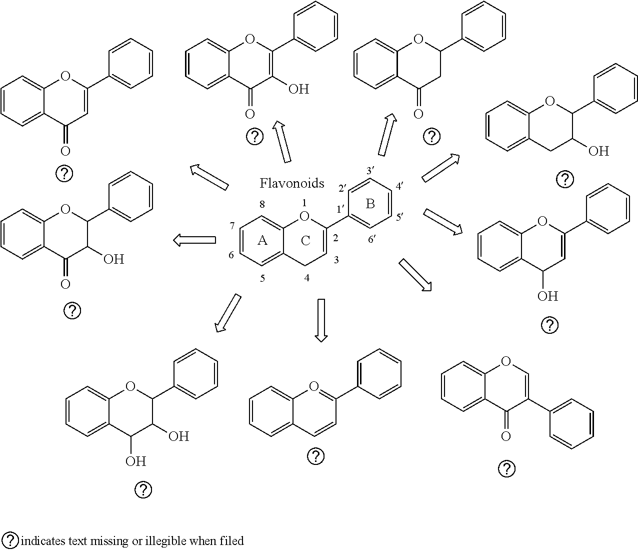

Preferred flavonoid compounds of Formula I include:
EGCG (CAS #989-51-5), EC (CAS #490-46-0), EGC (CAS #970-74-1) or ECG (CAS #1257-08-5)

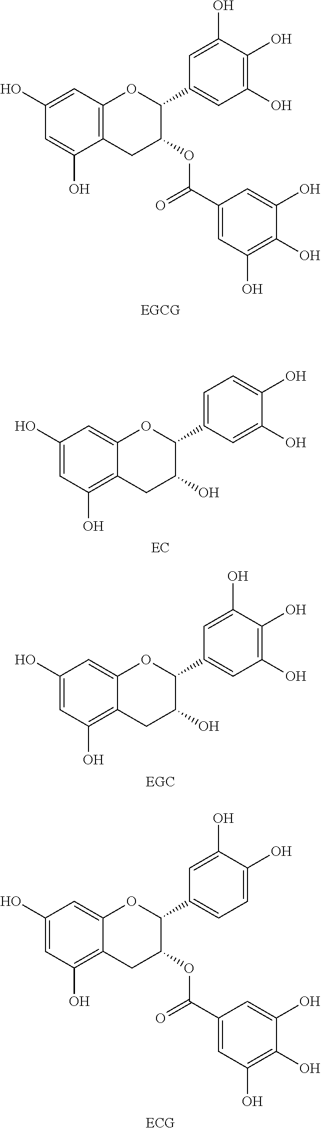 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]

### Conjugate

The present invention provides a conjugate comprising: (a) a cancer-targeting ligand, (b) a hydrophilic polymer of polyethylene glycol (PEG), polylactic acid (PLA), polylactic-co-glycolic acid (PLGA), or dextran, and (c) a flavonoid of Formula I, wherein the PEG covalently binds to the flavonoid and the cancer-targeting ligand.

The conjugate targets cancer by the cancer-targeting ligand, and delivers active ingredients to the cancer tissue to treat a cancer

The cancer-targeting ligand is covalently linked to PEG, PLA, PLGA, or dextran, either through its —COOH groups or its —NH₂ groups by a standard chemistry known to a person skilled in the art. The molecular weight of the hydrophilic polymer in the conjugate is in general 1K-100K, preferably 3K-80K, and more preferably 5K-40K.

The flavonoid in the conjugate has a general formula (I), and is preferably EGCG, EC, EGC, or ECG. In one embodiment, the flavonoid is epigallocatechin gallate (EGCG).

In one embodiment, PEG contains an aldehyde group which is conjugated to the 5, 6, 7, or 8 position (preferably 6 or 8 position) of the A ring of the flavonoid compound.

In another embodiment, PEG contains a thiol group which is conjugated to R₁ or R₂ of the B-ring of a flavonoid (when R₁ or R₂ is —OH).

In one embodiment, the conjugate comprises PEG-EGCG, which is PEG linked to one or two molecules of EGCG; which can be prepared by conjugating aldehyde-terminated PEG to EGCG by attachment of the PEG via reaction of the free aldehyde group with the 5, 6, 7, or 8 position (preferably 6 or 8 position) of Formula I. See WO2006/124000 and WO2009/054813. PEG-EGCG can also be prepared by conjugating thio-terminated PEG to EGCG by attachment of the PEG via reaction of the free thio group with the R₁ or R₂ of Formula I, wherein, R₁ or R₂ is a phenyl group. See WO2015/171079.

In another embodiment, the conjugate comprises PEG-EC, PEG-EGC, or PEG-ECG, and the conjugate can be prepared by conjugating aldehyde-terminated PEG to EC, EGC, or ECG by attachment of the PEG via reaction of the free aldehyde group with the 5, 6, 7, or 8 position (preferably 6 or 8 position) of Formula I.

HOOC-PEG-CHO and HO-PEG-CHO are commonly available. In one embodiment, HOOC-PEG-CHO is conjugated to EGCG, EC, EGC, or ECG according to WO2006/124000 and WO2009/054813. HOOC-PEG-flavonoid has COOH group to react with the N terminal of a cancer-targeting peptide. In general, a cancer-targeting peptide is incubated with HOOC-PEG-flavonoid, N, N′-dicyclohexylcarbodiimide (DCC), and N-Hydroxysuccinimide (NHS) in DMSO. The reaction is stirred at room temperature, prevented from light, under nitrogen. The reaction mixture is dialyzed (membrane Mw cutoff=2000 Da) against methanol and distilled water. Next, the solution is freeze-dried to obtain lyophilized powder. To avoid self-reaction of the peptide, the C terminal of the cancer-targeting peptide may be protected, for example, by resin during the reaction. Merrifield, hydroxymethyl polystyrene, PAM and MBHA resins are commonly used for preventing unwanted peptide conjugation. After the reaction, the resin can be removed under acidic condition.

In another embodiment, HO-PEG-CHO is conjugated to EGCG, EC, EGC, or ECG according to WO2006/124000 and WO2009/054813. HO-PEG-flavonoid has OH group to react with the C terminal of a cancer-targeting peptide. In general, a peptide is incubated with HO-PEG-EGCG, and N, N′-dicyclohexylcarbodiimide (DCC) in DMSO. The reaction is stirred at room temperature, prevented from light, under nitrogen. The reaction mixture is dialyzed (membrane Mw cutoff=2000 Da) against methanol distilled water. Next, the solution is freeze-dried to obtain lyophilized powder. To avoid self-reaction of the peptide, the N terminal of the cancer-targeting peptide may be protected, for example, by resin during the reaction. Merrifield, hydroxymethyl polystyrene, PAM and MBHA resins are commonly used for preventing unwanted peptide conjugation. After the reaction, the resin can be removed under acidic condition. In this reaction, COOH group on the peptide selectively reacts with OH on PEG, because the primary OH group on PEG is more reactive than the tertiary OH in the aromatic ring of flavonoid.

HOOC-PLA-CHO, HOOC-PLGA-CHO, and HO-Dextran-CHO are commercially available.

In one embodiment, HOOC-PLA-CHO is conjugated to EGCG, EC, EGC, or ECG according to WO2006/124000 and WO2009/054813. HOOC-PLA-flavonoid has COOH group to react with the N terminal of a cancer-targeting peptide. In general, a cancer-targeting peptide is incubated with HOOC-PLA-flavonoid, N, N′-dicyclohexylcarbodiimide (DCC), and N-Hydroxysuccinimide (NHS) in DMSO. The reaction is stirred at room temperature, prevented from light, under nitrogen. The reaction mixture is dialyzed (membrane Mw cutoff=2000 Da) against methanol and distilled water. Next, the solution is freeze-dried to obtain lyophilized powder. To avoid self-reaction of the peptide, the C terminal of the cancer-targeting peptide may be protected, for example, by resin during the reaction. Merrifield, hydroxymethyl polystyrene, PAM and MBHA resins are commonly used for preventing unwanted peptide conjugation. After the reaction, the resin can be removed under acidic condition.

In one embodiment, HOOC-PLGA-CHO is conjugated to EGCG, EC, EGC, or ECG according to WO2006/124000 and WO2009/054813. HOOC-PLGA-flavonoid has COOH group to react with the N terminal of a cancer-targeting peptide. In general, a cancer-targeting peptide is incubated with HOOC-PLGA-flavonoid, N, N′-dicyclohexylcarbodiimide (DCC), and N-Hydroxysuccinimide (NHS) in DMSO. The reaction is stirred at room temperature, prevented from light, under nitrogen. The reaction mixture is dialyzed (membrane Mw cutoff=2000 Da) against methanol and distilled water. Next, the solution is freeze-dried to obtain lyophilized powder. To avoid self-reaction of the peptide, the C terminal of the cancer-targeting peptide may be protected, for example, by resin during the reaction. Merrifield, hydroxymethyl polystyrene, PAM and MBHA resins are commonly used for preventing unwanted peptide conjugation. After the reaction, the resin can be removed under acidic condition.

In one embodiment, HO-Dextran-CHO is conjugated to EGCG, EC, EGC, or ECG according to WO2006/124000 and WO2009/054813. HO-Dextran-flavonoid has OH group to react with the C terminal of a cancer-targeting peptide. In general, a peptide is incubated with HO-Dextran-EGCG, and N, N′-dicyclohexylcarbodiimide (DCC) in DMSO. The reaction is stirred at room temperature, prevented from light, under nitrogen. The reaction mixture is dialyzed (membrane Mw cutoff=2000 Da) against methanol distilled water. Next, the solution is freeze-dried to obtain lyophilized powder. To avoid self-reaction of the peptide, the N terminal of the cancer-targeting peptide may be protected, for example, by resin during the reaction. Merrifield, hydroxymethyl polystyrene, PAM and MBHA resins are commonly used for preventing unwanted peptide conjugation. After the reaction, the resin can be removed under acidic condition. In this reaction, COOH group on the peptide selectively reacts with OH in the CH₂OH terminal of dextran, because this is the only primary OH group in dextran, which is more reactive than other secondary OH in dextran and tertiary OH in the aromatic ring of flavonoid.

The cancer-targeting ligand in the present invention is a ligand selected to target receptors on a cancer cell surface or tumor environment. The cancer-targeting ligands of the present invention, for example, target the following receptors on cancer, including but not limited, to integrin receptors (including α1β1, α2β1, α3β1, α4β1, α5β1, α6β1, α7β1, αLβ2, αMβ2, ICAM-1, αIIbβ3, αVβ1, αVβ3, αVβ3αVβ5αVβ5 αVβ3, αVβ5, αVβ5, αVβ6, αVβ8, α6β4), transferrin receptor, FGFR1, FGFR2, FGFR3, FGFR4, EGFR1 (HER1), EGFR2 (HER2), EGFR3 (HER3), EGFR 4 (HER4), TNFR1, TNFR2, c-MET (HGFR), NOTCH1, NOTCH2, NOTCH3, NOTCH4, IR, AR, ER, PRPTK7 receptor, TrkA, TrkB. TrkC, GPCR, Eph receptors, AXL receptor, Frizzled receptors, RET, ROS, folate receptor, IL-2R, IL-2RG, IL-2RB, IL-4R, IL-7R, IL-9R, CD133 receptor, LHRHR, LRP5, LRP6, CD44 receptor, CD47 receptor, CD20 receptor, Fas receptor, DR4, DR5, LEP-R, MUC1 receptor, adiponectin receptor, α-Adrenergic receptor, nucleolin receptor, PD-L1, ASGPR, lectin receptor, annexin receptor, glycyrrhetinic acid receptor cadherin and EpCAM. The cancer-targeting ligands of the present invention, for example, interact with the following targets in tumor microenvironment, the targets including but not limited to α1β1, α2β1, α3β1, α4β1, α5β1, α6β1, α7β1, αLβ2, αMβ2, ICAM-1, αIIbβ3, αVβ1, αVβ3, αVβ3αVβ5αVβ5 αVβ3, αVβ5, αVβ5, αVβ6, αVβ8, α6β4MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, MMP28, CCL2, CCL5, CXCL12, ICAM-1, VEGF-A, VEGF-B, VEGF-C, VEGF-D, PIGF, CCR2, CXCR4, CSF-1R, VEGFR1, VEGFR2, VEGFR3, FGF1, FGF2, FGF3, FGF4, FGF5, FGF6, FGF7, FGF8, FGF9, FGF10, FGF11, FGF12, FGF13, FGF14. FGF15, FGF16, FGF17, FGF18, FGF19, FGF20, FGF21, FGF22, FGF23, WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11, WNT16, IL-1, IL-2, IL-3, IL-4, IL-5, IL-6, IL-7, IL-8, IL-9, IL-10, IL-11, IL-12, IL-13, IL-15, IL-17, IL-21, IL-1R, IL-2R, IL-3R, IL-4R, IL-5R, IL-6R, IL-7R, IL-8R, IL-9R, IL-1R, IL-11R, IL-12R, IL-13R, IL-15R, IL-17R, IL-21R, PDGF, PDGFR-α, PDGFR-β, IGF1, IGF2, TGF-β, EGF, HB-EGF, TNF-α, TNFβ, TNFγ, CD19, CD103, IDO, PD-1, CTLA-4, collagens, elastin, glycosaminoglycans, proteoglycans, cancer associated fibroblast, tumor associated macrophage, T cell, B cell and dendritic cell.

In one embodiment, the cancer-targeting ligand is RGD, which targets integrin αvβ3.

In one embodiment, the cancer-targeting ligand is TP12 having the amino acid sequence of THRPPMWSPVWP (SEQ ID NO: 1), which targets transferrin receptor on various cancers.

In one embodiment, the cancer-targeting ligand is MC11 having the amino acid sequence of MQLPLATGGGC (SEQ ID NO: 2), which targets FGFR on various cancers.

In one embodiment, the cancer-targeting ligand is FV12 having the amino acid sequence of FCDGFYACYMDV (SEQ ID NO: 3), which targets HER2 on various cancers.

In one embodiment, the cancer-targeting ligand is YI12 having the amino acid sequence of YHWYGYTPQNVI (SEQ ID NO: 4), which targets EGFR

In one embodiment, the cancer-targeting ligand is CTT having the amino acid sequence of CTTHWGFTLC (SEQ ID NO: 5), which targets MMP2/MMP9 in tumor microenvironment

In one embodiment, the cancer-targeting ligand is H2009.1 peptide having the amino acid sequence of RGDLATLRQLAQEDGVVGVR (SEQ ID NO: 6), which targets Integrin αvβ6 receptor.

In one embodiment, the cancer-targeting ligand is IL-13 peptide having the amino acid sequence of GSETWKTIITKN (SEQ ID NO: 7), which targets IL-13Rα2 receptor.

In one embodiment, the cancer-targeting ligand is AP-1 peptide having the amino acid sequence of RKRLDRN (SEQ ID NO: 8), which targets IL-4 receptor.

In one embodiment, the cancer-targeting ligand is CVKTPAQSC (SEQ ID NO: 9), which targets CD133+ receptor.

In one embodiment, the cancer-targeting ligand is CC9 peptide having the amino acid sequence of CDCRGDCFC (SEQ ID NO: 10), which targets integrin in cancer and tumor environment including αvβ3, αvβ5, αvβ6, αvβ8, αIIbβ3, α8β1, and α5β1.

In one embodiment, the cancer-targeting ligand is RGDS peptide having the amino acid sequence of RGDS (SEQ ID NO: 11), which targets Integrin αvβ3 receptor.

In one embodiment, the cancer-targeting ligand is NR7 peptide having the amino acid sequence of NSVRGSR (SEQ ID NO: 12), which targets

In one embodiment, the cancer-targeting ligand is LHRH peptide having the amino acid sequence of EHWSYGLRPG (SEQ ID NO: 13), which targets LHRHR.

In one embodiment, the cancer-targeting ligand is angiopep-2 peptide having the amino acid sequence of TFFYGGSRGKRNNFKTEEY (SEQ ID NO: 14), which targets LRP.

In one embodiment, the cancer-targeting ligand is TbFGF peptide having the amino acid sequence of KRTGQYKLC (SEQ ID NO: 15), which targets EGFR.

In one embodiment, the cancer-targeting ligand is EGF peptide having the amino acid sequence of YHWYGYTPQNVI (SEQ ID NO: 16), which targets EGFR.

In one embodiment, the cancer-targeting ligand is folic acid or folate (C₁₉H₁₉N₇O₆), which targets folate receptor in cancer.

In one embodiment, the cancer-targeting ligand is hyaluronic acid, which targets CD44 receptor.

In one embodiment, the cancer-targeting ligand is lactose or galactosamine, which targets ASGPR.

In one embodiment, the cancer-targeting ligand is galactose, which targets ASGPR and lectin receptor.

In one embodiment, the cancer-targeting ligand is glycyrrhetinic acid, which targets glycyrrhetinic acid receptor.

### Nanoparticle Composition

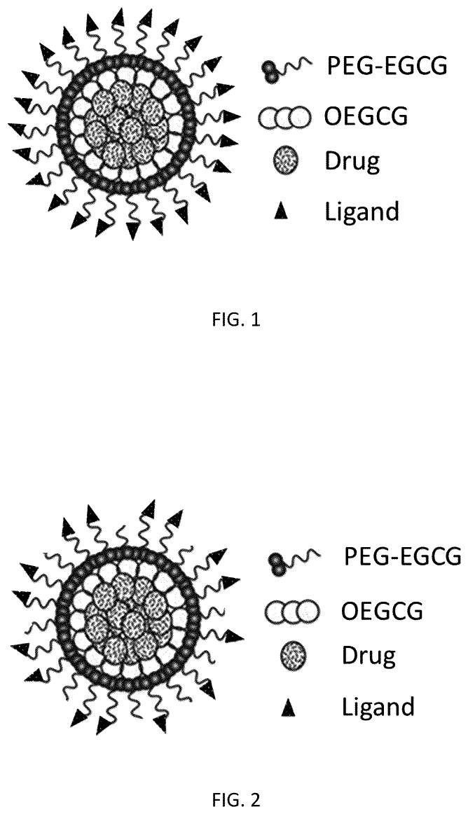

The term “MINC” (Multi-pathway Immune-modulating Nanocomplex Combination therapy) is a platform technology. The present invention provides a nanoparticle micelle (MINC) composition. The micelle comprises cancer-targeting ligand-PEG-flavonoid conjugate in an outer shell and oligomeric EGCG (OEGCG) in an inner shell (see FIG. 1). The cancer-targeting ligand allows the nanoparticle composition to specifically target the cancer tissues.

In one embodiment, the nanoparticle micelle composition has a defined and narrow size distribution in that at least 70% of the nanoparticles have a diameter between 20-500 nm or 50-300 nm, and the size distribution of the nanoparticles has only one major peak containing more than 90% of all the nanoparticles.

The micelles optionally comprise a cancer-treating molecule (an agent) encapsulated within the micelle (MINC-agent)

The present MINC-agent composition comprises three active ingredients, which are complementary in function to tackle both immune response and signaling pathways by its backbone components (PEG-flavonoid/OEGCG), and additional signaling pathways by a selected drug molecule for treating cancer. Each nanoparticle is a fixed-dose combination drug with the three active ingredients at fixed molar ratio.

The present invention delivers MINC-agent to targeted cancer tissues by active delivery of the micelles through a cancer-targeting ligand to tumors with specific receptors. In addition, the present invention delivers MICN-agent to tumors by passive delivery that relies on nanoparticle size.

The size of a drug product determines how much of the drug selectively goes to tumor versus unintended other tissues. Normal, unintended healthy tissues have blood vessel openings of less than 10 nm in general. A nanoparticle with a single size distribution around 100 nm can be uptaken by the targeted cells more efficiently. It has been demonstrated that the nanoparticle with single peak and restricted size around 100 nm can deliver more therapeutic agents to a targeted cell. The nanoparticle micelle composition of the present invention has a majority of the particles in the size of 20-500 nm or 50-300 nm. If the particle size is smaller than 50 nm, there is a higher risk that the particle will not be uptake well by the cell. If the particle size is greater than 300 nm, it may cause excess uptake by reticuloendothelial (RE) system and resulting in side effects. The present composition has more than 70% of the particles in the size range of 20-500 nm, which ensures the therapeutic agents enter the disease lesions preferentially over normal tissue and RE system. In addition, the restricted nanoparticle size of 20-500 nm can be selectively uptaken by a targeted cell to increase the drug (the agent) efficacy.

The nanoparticle micelle composition of the present invention has a narrow particle size distribution in that it has only one major peak that contains more than 90% of all the nanoparticles. It is important to have a therapeutic composition having only one peak of particle size distribution, instead of several peaks or multiple peaks. If a therapeutic composition has more than a single molecular size, it can cause severe variations in therapeutic efficacy and patient response rate.

The nanocomplex of the present invention contains the first two active ingredients, OEGCG and PEG-flavonoid such as PEG-EGCG in the backbone of the micelle composition. They are derivatives of EGCG, which is a strong immune modulator and regulates a wide spectrum of disease signaling pathways. EGCG regulates both innate and adaptive immunity. However, the bioavailability of EGCG is low and EGCG is not stable. The present nanocomplex composition overcomes the bioavailability issue of EGCG by forming a nanocarrier to carry EGCG to a target site for treatment, and overcomes the stability issue of EGCG by forming OEGCG and PEG-EGCG complex, which effectively enables EGCG as highly effective therapeutic agents.

The nanocomplex of the present invention optionally contains a third active ingredient, which is a drug molecule encapsulated in the nanoparticles for treating cancer.

Nanoparticles with sizes 20-500 nm preferentially go to intended treatment site. Large nanoparticles (500-999 nm) or micron-size (1000-5000 nm) particles, due to aggregation of smaller nanoparticles, may lead to toxicity because large nanoparticles are often efficiently taken up by the reticuloendothelial system (RES), also known as the mononuclear phagocytic system (MPS) located in the liver, lungs, and bone marrow.

The inventors have discovered a nanoparticle micelle composition of the present invention for the target delivery to target tissue, with at least 70% of the nanoparticles having a diameter between 20-500 nm or 50-300 nm, and the size distribution of the nanoparticles only has one major peak that contains more than 90% of all the particles. The inventors have also discovered a process for preparing such nanoparticle composition.

The present invention is directed to a nanoparticle composition comprising nanoparticles having: (a) an inner core comprising oligomeric (−)-epigallocatechin gallate (OEGCG), (b) an outer core comprising a cancer-targeting ligand-PEG-flavonoid conjugate, and (c) a drug molecule encapsulated in the inner core. In one embodiment, at least 70% of the nanoparticles have a diameter between 20-500 nm or 50-300 nm, and the size distribution of the nanoparticles only has one major peak that contains more than 90% of all the particles. The flavonoid in the conjugate is preferably EGCG, EC, EGC, or ECG, with EGCG being more preferred. The structure of the nanoparticles of the present invention is shown in FIG. 1.

In one embodiment, the micelle outer shell further comprises a bare PEG-flavonoid conjugate such as PEG-EGCG, which does not have a cancer-targeting ligand linked to PEG-flavonoid. In such a micelle outer shell, the ratio of ligand-PEG-EGCG to ligand-PEG-EGCG plus PEG-EGCG is in general more than 10%, or more than 20%, or more than 30%, or more than 50%, and up to 100%. See FIG. 2.

In one embodiment, at least 80%, or at least 85%, or at least 90%, or at least 95% of the nanoparticles have a diameter between 20-500 nm or 50-300 nm.

In one embodiment, the median nanoparticle diameter in the nanoparticle composition is between 50 to 250 nm, 50 to 200 nm, 80 to 200 nm, 100 to 200 nm, or 50 to 150 nm.

In one embodiment, the size distribution of the nanoparticles only shows one major and narrow peak that contains more than 80%, more than 85%, more than 90%, more than 95%, or more than 98% of all the particles.

In one embodiment, the cancer-treating drug molecule in MINC-agent is a cytokine, an antibody, a chemotherapy agent, or a small compound inhibitor.

Suitable cancer-treating cytokines for the present invention include, but are not limited to, IL-2, IL-6 IL-7, IL-10, IL-12, IL-15, IL-17, IL-21, TARIL, IGF1, GLP-1, IFN-α, IFN-β, IFN-γ, CCL5, CXCL9, CXCL10, CXCL11, CX3CL1, and recombinant cytokine products.

Suitable cancer-treating antibodies for the present invention include, but are not limited to, monoclonal antibody, polyclonal antibody, antibody-drug-conjugate, and bispecific antibody. Preferred antibodies for the present invention are monoclonal antibodies. Antibodies suitable for the present invention include anti-PD-1 antibody, anti-PD-L1 antibody, anti-CTLA-4 antibody, anti-LAG3 antibody, anti-TIGIT antibody, anti-TIM3 antibody, anti-HER2 antibody, anti-HER3 antibody, anti-HGFR antibody, anti-EGFR antibody, anti-EpCAM, anti-FOLR1 antibody, anti-c-Met antibody, anti-GD2 ganglioside antibody, anti-GD3 ganglioside, anti-VEGFR1 antibody, anti-VEGF antibody, anti-TGF-β antibody, anti-TNF-α antibody, anti-IGF-1R antibody, anti-IL-4 antibody, anti-IL-10 antibody, anti-IL-13 antibody, anti-CD3 antibody, anti-CD4 antibody, anti-CD40 antibody, anti-CD40L antibody, anti-CD43 antibody, anti-CD19 antibody, anti-CD27 antibody, anti-CD70 antibody, anti-CD71 antibody, anti-CD28 antibody, anti-CD38 antibody, anti-CD20 antibody, anti-B7-H3 antibody, anti-B7-H4 antibody, anti-DR5 antibody, anti-MUC1 antibody, anti-Tau antibody, anti-amyloid antibody, abagovomab, abituzumab, adalimumab, aducanumab, alemtuzumab, amatuximab, amivantamab, anifrolumab, atezolizumab, avelumab, bapineuzumab, basiliximab, belimumab, benralizumab, besilesomab, bevacizumab, bezlotoxumab, blinatumomab, brazikumab, brontictuzumab, cabiralizumab, camrelizumab, carlumab, carotuximab, catumaxomab, cedelizumab, cetrelimab, cetuximab, cibisatamab, crenezumab, cusatuzumab, daclizumab, daclizumab, dalotuzumab, daratumumab, detumomab, dinutuximab, drozitumab, duligotuzumab, dupilumab, durvalumab, ecromeximab, emibetuzumab, epcoritamab, epratuzumab, eptinezumab, erenumab, ertumaxomab, etaracizumab, etesevimab, farletuzumab, fezakinumab, ficlatuzumab, figitumumab, fletikumab, foralumab, fresolimumab, futuximab, ganitumab, gantenerumab, gatipotuzumab, gevokizumab, golimumab, guselkumab, icrucumab, igovomab, imalumab, imgatuzumab, inebilizumab, infliximab, intetumumab, ipilimumab, istiratumab, ixekizumab, letolizumab, lexatumumab, lintuzumab, mapatumumab, matuzumab, mavrilimumab, mepolizumab, mogamulizumab, monalizumab, mosunetuzumab, natalizumab, naxitamab, necitumumab, nimotuzumab, nivolumab, ocaratuzumab, ocrelizumab, ofatumumab, olaratumab, olaratumabopicinumab, panitumumab, pembrolizumab, pertuzumab, ponezumab, ramucirumab, ranibizumab, rituximab, samalizumab, sarilumab, secukinumab, sintilimab, solanezumab, teprotumumab, tigatuzumab, tildrakizumab, timigutuzumab, tocilizumab, tomuzotuximab, trastuzumab, ustekinumab, vanucizumab, varisacumab, varlilumab, vedolizumab, vepalimomab, vesencumab, visilizumab, vonlerolizumab, zanolimumab, zatuximab, zenocutuzumab, zolbetuximab, ado-trastuzumab emtansine, anetumab ravtansine, brentuximab vedotin, cantuzumab mertansine, certolizumab pegol, coltuximab ravtansine, depatuxizumab mafodotin, enapotamab vedotin, gemtuzumab ozogamicin, glembatumumab vedotin, iladatuzumab vedotin, inatuzumab vedotin, indatuximab ravtansine, indusatumab vedotin, lifastuzumab vedotin, lilotomab satetraxetan, lorvotuzumab mertansine, losatuxizumab vedotin, lulizumab pegol, mirvetuximab soravtansine, naratuximab emtansine, notuzumab ozogamicin, polatuzumab vedotin-piiq, rovalpituzumab tesirine, sacituzumab govitecan, samrotamab vedotin, telisotuzumab vedotin, trastuzumab deruxtecan, and tucotuzumab celmoleukin. Antibodies also include antibody fragments that are capable of binding to their corresponding antigens, such as Fab, (Fab)2, or single-chain antibodies.

Suitable cancer-treating chemotherapy agent for the present invention include, but are not limited to altretamine, busulfan, carboplatin, carmustine, cisplatin, cyclophosphamide, dacarbazine, ifosfamide, lomustine, melphalan, temozolomide, trabectedin, 5-fluorouracil, 6-mercaptopurine, azacitidine, capecitabine, clofarabine, cytarabine, floxuridine, fludarabine, gemcitabine, methotrexate, pemetrexed, pentostatin, pralatrexate, trifluridine, vincristine, vinblastine, vinorelbine, paclitaxel, docetaxel, etoposide, teniposide, irinotecan, topotecan, daunorubicin, doxorubicin, epirubicin, idarubicin, and valrubicin.

Suitable cancer-treating small compound inhibitors for the present invention include but not limited to imatinib, gefitinib, erlotinib, sunitinib, lapatinib, nilotinib, sorafenib, temsirolimus, everolimus, pazopanib, crizotinib, ruxolitinib, vandetenib, axitinib, bosutinib, cabozantinib, ponatinib, regorafenib, ibrutinib, trametinib, or perifosine, for targeting tyrosine, and serine/threonine kinases.

Suitable cancer-treating small compound inhibitors for the present invention include but not limited to bortezomib, carfilzomib, or marizomib, for targeting proteasomes.

Suitable cancer-treating small compound inhibitors for the present invention include but not limited to batimastat, neovastat, prinomastat, rebimastat, marimastat, ganetespib, or NVP-AUY922, for targeting MMPs and HSPs.

Suitable cancer-treating small compound inhibitors for the present invention include but not limited to Obatoclax or Navitoclax, for inducing apoptosis.

The nanoparticle composition of the present invention has a majority of particle size of 20-500 nm or 50-300 nm in diameter with OEGCG, PEG-EGCG, and a drug molecule held together by hydrophobic interactions. It is stable in a hydrophilic environment, such as blood circulation, and dissociates in a hydrophobic environment, such as a tumor tissue. It can selectively diffuse from blood vessels to surrounding tissue with leaky vessels due to inflammation and other hyperactivities, such as rapid, uncontrolled tumor growth. Due to its size, it is restricted from entering normal tissues with less leaky vessels. Once the nanoparticle complex enters tissue which is hydrophobic, it dissociates and frees its active components OEGCG, PEG-flavonoid such as PEG-EGCG, and the drug molecule in the nanocomplex. The free active components regain their bioactivities in cancer retardation. The active components in the nanoparticles have a longer circulation half-life and act as a slow-release mechanism which further lowers the drug dosage requirement. Consequently, any adverse effects to normal tissues are further diminished.

The MINC-agent composition further has the following advantages:
MINC is stable for >15 days in blood circulation. The encapsulated drug molecules are protected within the MINC nanoparticle shell during circulation.
MINC nanoparticle uses enhanced permeability and retention (EPR) effect and sends majority of the drug molecules to target cells.
MINC nanoparticle have no aggregations during the freeze-thaw cycle.
MINC drugs retain their original biological activities after lyophilization.
MINC drugs are stable at 2-8° C.

### Process for Preparing the Nanoparticle Composition

The present invention provides a process for preparing nanoparticle composition of a fixed-dose combination drug. The process is optimized so only the nanometer-size particles with at least 70% of the particles having a diameter between 20-500 nm or 50-300 nm and one major peak are produced.

The process comprises the steps of: (a) mixing a drug molecule with OEGCG and cancer-targeting ligand-PEG-EGCG conjugate in an aqueous solution; and (b) filtering the mixture through a membrane with a molecular weight cut-off of 8,000-300,000 daltons to remove small molecular weight molecules and retain large molecular weight molecules.

In one preferred embodiment, the process further comprises step (c), filtering the large molecular weight molecules through 0.2-0.3 μm membrane and collecting the filtrate.

The present process optionally further comprises a lyophilization step (d) after step (c). Step (d): lyophilizing the filtrate by stepwise freezing at (i) about 0-5° C., (ii) about −20 to −30° C., and (iii) at about −60 to −100° C., and then drying.

In step (a), the drug molecule is dissolved in an aqueous solvent, such as phosphate-buffer saline, saline, water, bicarbonate buffer, oxyhemoglobin buffer, bis-tris alkane, Tris-HCl, HEPES, histidine buffer, NP-40, RIPA (radioimmunoprecipitation assay buffer), tricine, TES, TAPS, TAPSO, Bicine, MOPS, PIPES, cacodylate, or MES. Preferred solvents are phosphate-buffer saline, saline, or water. The protein drug concentration is in general 0.01-50 mg/ml, preferred 0.05-10 mg/ml, and more preferred 0.1-5 mg/ml.

OEGCG, PEG-EGCG, and optionally EGCG, are dissolved in ketones, acetonitrile, alcohols, aldehydes, ethers, acetates, sulfoxides, benzenes, organic acids, amides, aqueous buffers, and any combination thereof. Preferred solvents are alcohols, acetonitrile, sulfoxides, amides, and any combination thereof. The OEGCG/EGCG and PEG-EGCG concentrations are in general independently 0.001-10 mg/ml, preferred 0.005-1 mg/ml, or 0.1-5 mg/ml.

It is important that OEGCG is in molar excess of the drug agent. In general, the molar ratio of the EGCG in OEGCG to the drug molecule is between 1-500 to 1, 2-500 to 1, 3-500 to 1, or 5-500 to 1, preferably 3-100 to 1, 5-100 to 1, or 10-50 to 1. The molar ratio is calculated by the number of moles of monomer EGCG in OEGCG to the number of moles of the drug molecule. The molar excess of EGCG ensures most or all drug agents are encapsulated by the OEGCG molecules. Unencapsulated drug agents, which would not be selectively distributed to target tissue and would cause lower efficacy and safety issues, are avoided by controlling the molar ratio of OEGCG to protein in the present process.

The drug agent, OEGCG, and PEG-EGCG are mixed between 1 minute to 2 days, preferably 1 minute to 12 hours, at a temperature between about 0° C. to 60° C., preferably 0° C. to 45° C., or 0° C. to 37° C.

In step (b), the above mixture is filtered through a membrane with a molecular weight cut-off between 8,000-300,000 daltons, preferably between 8,000-200,000 daltons, 8,000-150,000 daltons, or 8,000-12,000 daltons, to remove small molecular weight molecules and retain large molecular weight molecules. The ultrafiltration membrane material is selected from the group consisting of cellulose (and its derivatives), polyethersulfone (PES), polytetrafluoroethylene (PTFE), nylon, polyvinylidene fluoride or polyvinylidene difluoride (PVDF), and polypropylene (PP); preferably cellulose (and its derivatives), PTFE, and PVDF.

The mixture is optionally diluted in an aqueous solvent such as those described above in step (a) before ultrafiltration.

The ultrafiltration step (b) removes unwanted impurities of small molecular weight, such as unreacted OEGCG or EGCG, or reaction by-products. These impurities may reduce drug efficacy and safety. The excess of unreacted OEGCG or EGCG may also lead to aggregation of the individual nanoparticles to about 1000 nm size particles, which would reduce efficacy and cause potential toxicity.

In step (c), the retained large molecular weight molecules are filtered through a membrane having a pore size of about 0.2-0.3 μm, such as 0.22 μm, and the filtrate is collected. This is to remove unwanted impurities of large molecular sizes, such as mega-aggregates. These aggregates may be excreted from entering tissues due to its mega size. These aggregates reduce overall efficacy/safety and have a higher chance of inducing immunogenicity to the patients. Large size nanoparticles are also easier to be taken up by RES in the liver, lungs, and more undesired organs.

The membrane material of step (c) is selected from the group consisting of cellulose (and its derivatives), PES, PTFE, nylon, PVDF, and PP; preferably cellulose (and its derivatives), PES, and PP.

In one embodiment, the steps (b) and (c) are repeated at least one time, for example, repeated 1, 2, 3, or 4 times before step (d), to effectively remove unwanted small molecule impurities and large aggregates.

After step (c), the filtrate is stored at 2-8° C., and is stable for at least 100 days.

The present process optionally further comprises a lyophilization step (d) after step (c) to provide a long-term stability of the nanoparticle composition.

In step (d), the filtrate collected after filtration through 0.2-0.3 μm membrane is lyophilized by first stepwise freezing the filtrate at (i) about 0-5° C., for example, for about 1-3 hours, (ii) about −25° C. to −30° C., for example, for about 1-3 hours, then freezing at (iii) −60° C. to −100° C. or −70° C. to −100° C., for example, for at least 8 hours.

After freezing, the material is lyophilized for 1 to 7 days.

Freezing and lyophilization often cause nanoparticles to form complexes and aggregates. These large particles may be too big to penetrate blood vessels and enter tissue environments. Consequently, efficacy and safety are lower, and immunogenicity may increase. To avoid these changes caused by lyophilization, the present process uses a stepwise freezing process, instead of a continuously freezing process (lowering temperature gradually and continuously during freezing), to preserve the nanoparticle size during lyophilization.

### Pharmaceutical Compositions

The present invention provides pharmaceutical compositions comprising the nanoparticle composition of the present invention and optionally one or more pharmaceutically acceptable carriers. The nanoparticle composition in a pharmaceutical composition in general is about 1-90%, preferably 20-90%, or 30-80% for a tablet, powder, or parenteral formulation. The nanoparticle composition in a pharmaceutical composition in general is 1-100%, preferably 20-100%, 50-100%, or 70-100% for a capsule formulation. The nanoparticle composition in a pharmaceutical composition in general is 1-50%, 5-50%, or 10-40% for a liquid suspension formulation.

In one embodiment, the pharmaceutical composition can be in a dosage form such as tablets, capsules, granules, fine granules, powders, suspension, patch, parenteral, injectable, or the like. The above pharmaceutical compositions can be prepared by conventional methods.

Pharmaceutically acceptable carriers, which are inactive ingredients, can be selected by those skilled in the art using conventional criteria. The pharmaceutically acceptable carriers may contain ingredients that include, but are not limited to, saline and aqueous electrolyte solutions; ionic and nonionic osmotic agents, such as sodium chloride, potassium chloride, glycerol, and dextrose; pH adjusters and buffers, such as salts of hydroxide, phosphate, citrate, acetate, borate, and trolamine; antioxidants, such as salts, acids, and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants, such as lecithin and phospholipids, including, but not limited to, phosphatidylcholine, phosphatidylethanolamine and phosphatidyl inositol; poloxamers and poloxamines; polysorbates, such as polysorbate 80, polysorbate 60, and polysorbate 20; polyethers, such as polyethylene glycols and polypropylene glycols; polyvinyls, such as polyvinyl alcohol and polyvinylpyrrolidone (PVP, povidone); cellulose derivatives, such as methylcellulose, hydroxypropyl cellulose, hydroxyethyl cellulose, carboxymethyl cellulose, and hydroxypropyl methylcellulose and their salts; petroleum derivatives, such as mineral oil and white petrolatum; fats, such as lanolin, peanut oil, palm oil, and soybean oil; mono-, di-, and triglycerides; polysaccharides, such as dextrans; and glycosaminoglycans, such as sodium hyaluronate. Such pharmaceutically acceptable carriers may be preserved against bacterial contamination using well-known preservatives, which include, but are not limited to, benzalkonium chloride, ethylene diamine tetra-acetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet, capsule, or parenteral formulation of the active compound may contain other excipients that have no bioactivity and no reaction with the active compound. Excipients of a tablet or a capsule may include fillers, binders, lubricants and glidants, disintegrators, wetting agents, and release rate modifiers. Examples of excipients of a tablet or a capsule include, but are not limited to, carboxymethylcellulose, cellulose, ethylcellulose, hydroxypropylmethylcellulose, methylcellulose, karaya gum, starch, tragacanth gum, gelatin, magnesium stearate, titanium dioxide, poly(acrylic acid), and polyvinylpyrrolidone.

For example, a tablet formulation may contain inactive ingredients, such as colloidal silicon dioxide, crospovidone, hypromellose, magnesium stearate, microcrystalline cellulose, polyethylene glycol, sodium starch glycolate, and titanium dioxide. A capsule formulation may contain inactive ingredients, such as gelatin, magnesium stearate, and titanium dioxide. A powder oral formulation may contain inactive ingredients, such as silica gel, sodium benzoate, sodium citrate, sucrose, and xanthan gum.

### Method of Treatment

The present invention is directed to a method for treating cancer, comprising the step of administering an effective amount of the nanoparticle composition of the present invention to a subject in need thereof.

“An effective amount,” as used herein, is the amount effective to treat a disease by ameliorating the pathological condition or reducing the symptoms of the disease.

Suitable cancers to be treated by the present invention include adrenocortical carcinoma, AIDS-related lymphoma, anal cancer, appendix cancer, grade I (anaplastic) astrocytoma, grade II astrocytoma, grade III astrocytoma, grade IV astrocytoma, atypical teratoid/rhabdoid tumor of the central nervous system, basal cell carcinoma, bladder cancer, breast cancer, bronchial cancer, bronchioalveolar carcinoma, Burkitt lymphoma, cervical cancer, colon cancer, colorectal cancer, craniopharyngioma, cutaneous T-cell lymphoma, endometrial cancer, endometrial uterine cancer, ependymoblastoma, ependymoma, esophageal cancer, esthesioneuroblastoma, Ewing's sarcoma, extracranial germ cell tumor, extragonadal germ cell tumor, extrahepatic bile duct cancer, fibrous histiocytoma, gallbladder cancer, gastric cancer, gastrointestinal carcinoid tumor, gastrointestinal stromal tumor, gestational trophoblastic tumor, gestational trophoblastic tumor, glioma, head and neck cancer, heart cancer, hepatocellular cancer, Hilar cholangiocarcinoma, Hodgkin's lymphoma, hypopharyngeal cancer, intraocular melanoma, islet cell tumor, Kaposi sarcoma, Langerhans cell histiocytosis, laryngeal cancer, lip cancer, lung cancer, lymphoma, macroglobulinemia, malignant fibrous histiocytoma, medulloblastoma, medulloepithelioma, melanoma, Merkel cell carcinoma, mesothelioma, endocrine neoplasia, multiple myeloma, mycosis fungoides, myelodysplasia, myelodysplastic/myeloproliferative neoplasms, myeloproliferative disorders, nasal cavity cancer, nasopharyngeal cancer, neuroblastoma, non-Hodgkin's lymphoma, oral cancer, oropharyngeal cancer, osteosarcoma, ovarian clear cell carcinoma, ovarian epithelial cancer, ovarian germ cell tumor, papillomatosis, paranasal sinus cancer, parathyroid cancer, penile cancer, pharyngeal cancer, pineal parenchymal tumor, pineoblastoma, pituitary tumor, plasma cell neoplasm, plasma cell neoplasm, pleuropulmonary blastoma, primary central nervous system lymphoma, prostate cancer, rectal cancer, renal cell cancer, respiratory tract cancer with chromosome 15 changes, retinoblastoma, rhabdomyosarcoma, salivary gland cancer, Sezary syndrome, small intestine cancer, soft tissue sarcoma, squamous cell carcinoma, squamous neck cancer, supratentorial primitive neuroectodermal tumor, supratentorial primitive neuroectodermal tumor, testicular cancer, throat cancer, thymic carcinoma, thymoma, thyroid cancer, cancer of the renal pelvis, urethral cancer, uterine sarcoma, vaginal cancer, vulvar cancer, Waldenstrom macroglobulinemia, and Wilms tumor.

Dosing for a ligand-PEG-EGGC conjugate, for injection, is in general 0.01-1200 mg/kg (total weight of the conjugate/subject body weight), or 0.1-1000 mg/kg.

Dosing of the MINC-agent is based on the known dosage of the agents for treating a particular disease and the subject condition. The dosage can be a food drug administration (FDA) approved dosage or a dosage used in clinical trial.

In MINC-agent, the weight of ligand-PEG-EGCG is closed to the encapsulated drug agent. In general, the dosage of PEG-EGCG combined with OEGCG is between 10 μg/kg to 100 mg/kg.

Dosing of the nanoparticle composition is based on the known dosage of the protein drug for treating a particular disease and the subject condition. For example, for treating breast cancer in an adult human, trastuzumab is administered 4-8 mg/kg via IV infusion once weekly for 52 weeks. The effective dose of ligand-MINC-trastuzumab is in the same dose range with a less frequent dosing frequency of once every 12 to 16 weeks for 52 weeks.

For example, for treating melanoma, interferon-α induction is 20 million IU/m² as an IV infusion, at 5 consecutive days per week for 4 weeks. The effective dose of ligand-MINC-interferon-α is in the same dosage range, administered at 1 day per week for 2 weeks to achieve the same efficacy and reduced toxicity.

For example, for treating kidney cancer, IL-12 at 600,000 International Units/kg (0.037 mg/kg) is administered three times a day for a maximum of 14 doses. Following 9 days of rest, the schedule is repeated for another 14 doses, as tolerated. The effective dose of ligand-MINC-IL-12 in the same dosage range is administered 1 dose a day for 3 days of 9 doses total.

The present invention is useful in treating human and non-human animals. For example, the present invention is useful in treating a mammal subject, such as humans, horses, pigs, cats, and dogs.

The following examples further illustrate the present invention. These examples are intended merely to be illustrative of the present invention and are not to be construed as being limiting.

### EXAMPLES

### Example 1: RGD Peptide Conjugation to HOOC-PEG-EGCG

### Materials

RGD peptide was purchased from BIOTOOLS

HOOC-PEG-CHO was purchased from NBC chemical

### Method

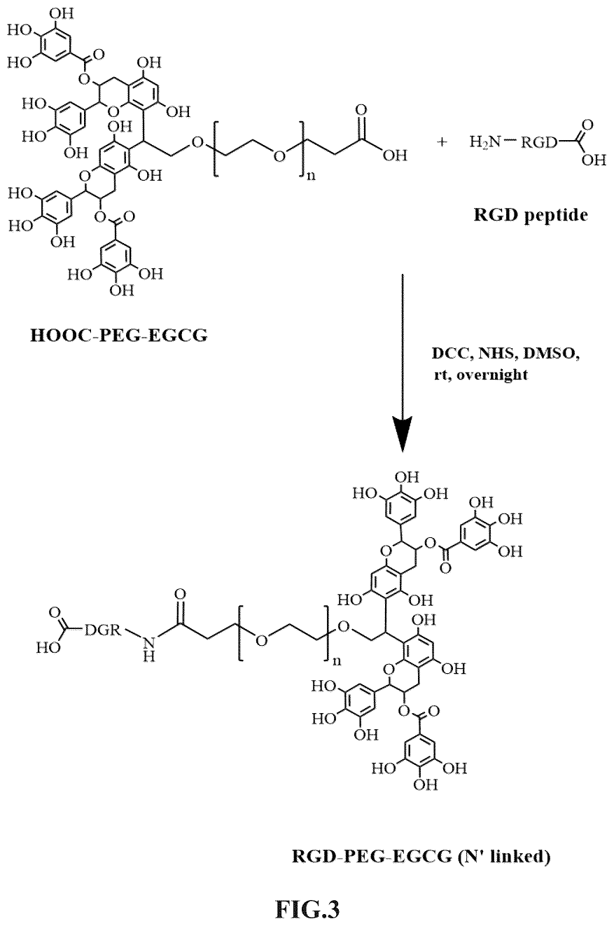

HOOC-PEG-CHO was conjugated to EGCG according to WO2006/124000 and WO2009/054813; RGD peptide was conjugated to HOOC-PEG-EGCG via the conjugation between COOH group on PEG and NH₂ group on RGD to form RGD-PEG-EGCG (N′ linked) (FIG. 3).

Specifically, 1-1000 mg RGD was PEGylated by incubation with 1-1000 mg HOOC-PEG-EGCG, 1-1000 mg N, N′-dicyclohexylcarbodiimide (DCC), and 1-1000 mg N-Hydroxysuccinimide (NHS) in DMSO. The reaction was stirred at room temperature, prevented from light, under nitrogen for 24 hours. The reaction mixture was dialyzed (membrane molecular weight cutoff=2000 Da) against methanol and distilled water for 3 days. Next, the solution was freeze-dried to obtain lyophilized powder.

### Results

HPLC was used to confirm the formulation of RGD-PEG-EGCG. HPLC was conducted under the following conditions: Column: C18, 4.6×150 mm, 4 m; Elution: A=0.1% TFA in H₂O, B=0.1% TFA in ACN; Oven temperature: 40° C.; Flow speed: 1 ml/min; Autosampler temperature: 15° C.; Measurement: UV280.

HOOC-PEG-EGCG had a retention time at 5.95 min, after RGD conjugation, a new peak with retention time at 6.38 min was present. The HPLC results indicate a successful conjugation of RGD-PEG-EGCG.

### Example 2: Transferrin Peptide (TP12) Conjugation to HOOC-PEG-EGCG

### Materials

TP12 peptide was purchased from BIOTOOLS

HOOC-PEG-CHO was purchased from NBC chemical

### Method

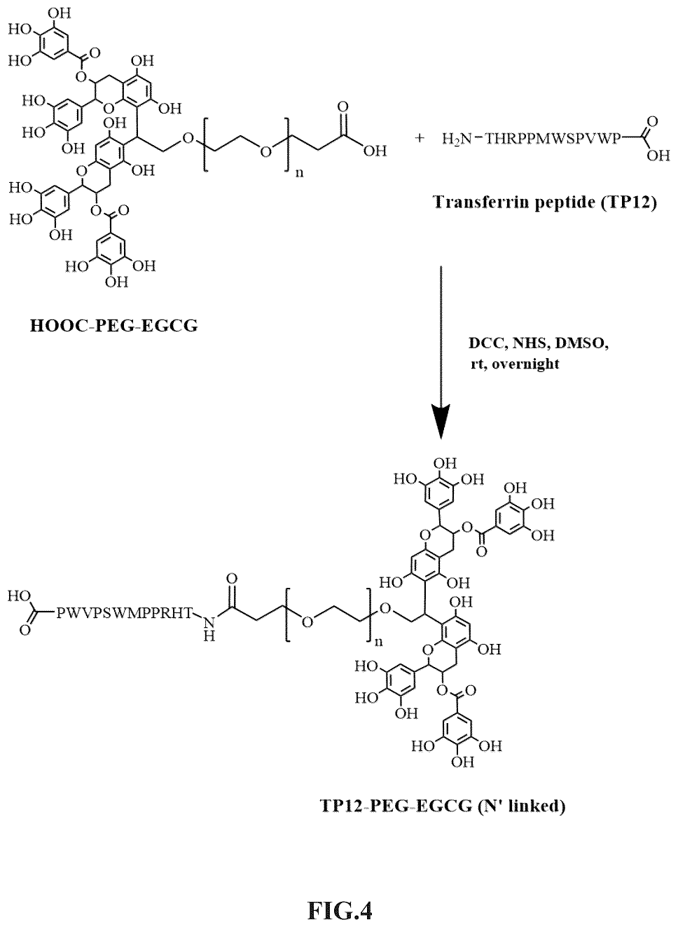

For TP12-PEG-EGCG formulation, HOOC-PEG-CHO was conjugated to EGCG according to WO2006/124000 and WO2009/054813; TP12 peptide was conjugated to HOOC-PEG-EGCG via the conjugation between COOH group on PEG and NH2 group on TP12 to form TP12-PEG-EGCG (N′ linked) (FIG. 4).

Specifically, 1-1000 mg TP12 was PEGylated by incubation with 1-1000 mg HOOC-PEG-EGCG, 1-1000 mg N, N′-dicyclohexylcarbodiimide (DCC), and 1-1000 mg N-Hydroxysuccinimide (NHS) in DMSO. The reaction was stirred at room temperature, prevented from light, under nitrogen for 24 hours. The reaction mixture was dialyzed (membrane Mw cutoff=2000 Da) against methanol and distilled water for 3 days. Next, the solution was freeze-dried to obtain lyophilized powder.

### Results

HPLC was used to confirm the formulation of TP12-PEG-EGCG. HPLC was conducted under the conditions: Column: C18, 4.6×150 mm, 4 μm; Elution: A=0.1% TFA in H2O, B=0.1% TFA in ACN; Oven temperature: 40° C.; Flow speed: 1 ml/min; Autosampler temperature: 15° C.; Measurement: ELSD].

HOOC-PEG-EGCG had a retention time at 6.04 min. After TP12 conjugation, a new peak with retention time at 6.28 min was present. The results indicate a successful conjugation of TP12-PEG-EGCG.

### Example 3: Preparing RGD-MINC-Doxorubicin and TP12-MINC-Doxorubicin

### Materials

RGD-PEG-EGCG was prepared according to Example 1.

TP12-PEG-EGCG was prepared according to Example 2.

Doxorubicin was purchased from Sigma-Aldrich or other suppliers.

### Method

RGD-MINC-doxorubicin nanoparticles and TP12-MINC-doxorubicin nanoparticles were prepared according to the following protocol:
1. Incubate 5-500 μg doxorubicin in 1 mL DMSO for 15 min to 1 hour.
2. Add 1-100 μg of OEGCG and 1-10,000 μg of RGD-PEG-EGCG or TP12-PEG-EGCG. Incubate the mixture at 25° C. for 3 hours.
3. Filter out the liquid with a 10K MWCO filter unit. Wash the filter 3 time with 0.9% NaCl
4. Lyophilize to dry powder.

### Results

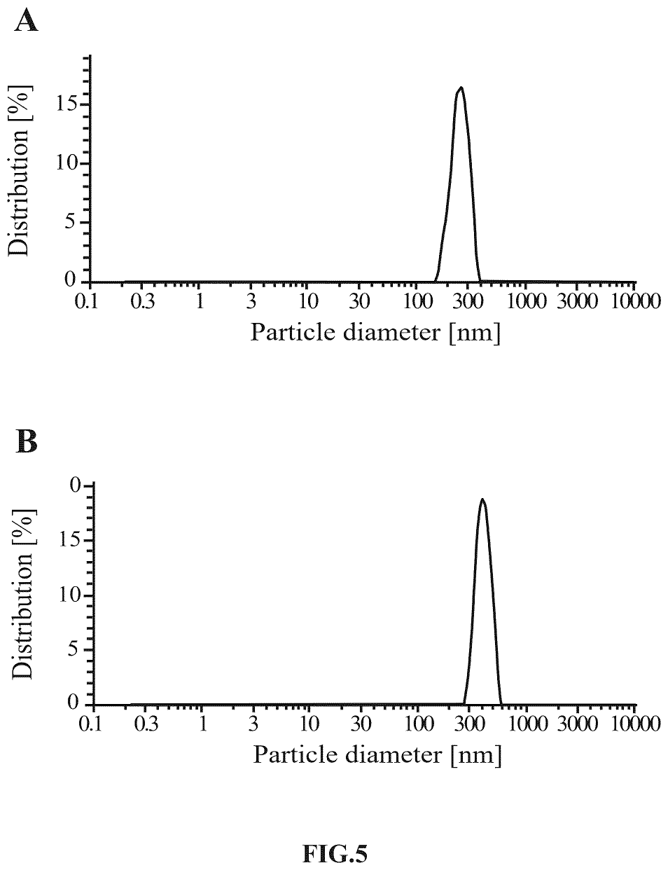

Nanoparticle size was measured by DLS (Anton Paar, Litesizer 500). FIG. 5 showed successful formulation of RGD-MINC-doxorubicin (A) and TP12-MINC-doxorubicin (B).

### Example 4: Different Ligand-MINC-Doxorubicin in Delivering Drugs into Cancer Cells

### Materials

RGD-MINC-doxorubicin, TfR-MINC-doxorubicin (TP12-MINC-doxorubicin) or MINC-doxorubicin were formulated according to Example 3.

### Method

Breast cancer cell line MDA-MB-231 was seeded at 1×10⁵ cells/well in 12 well plate with coverslip and incubated overnight. On the second day, cells were treated with MINC-doxorubicin, RGD-MINC-doxorubicin or TfR-MINC-doxorubicin at 2.5 μM for 2 and 24 hours. After the treatment, the cells were fixed with ice-cold methanol. The fluorescence image was taken by fluorescence microscope to assess the delivery efficiency. The fluorescent intensity was measured by Image J, using the parameter fixed area: 10.309. Higher fluorescence signal means more doxorubicin was uptaken by the cells.

### Result

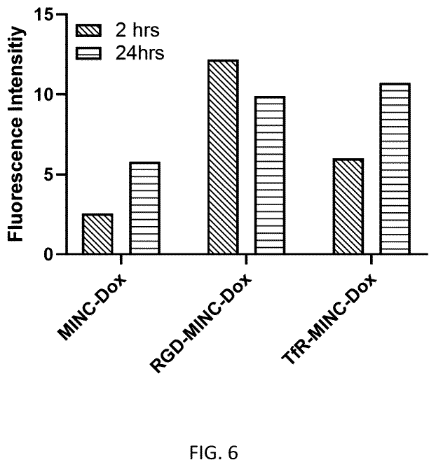

Doxorubicin is a red fluorescent compound, its delivery into cells was observed using fluorescence microscope. The higher fluorescence intensity means more doxorubicin was delivered into the cells. In FIG. 6, compared to MINC-doxorubicin, the fluorescent signal of RGD-MINC-doxorubicin or TfR-MINC-doxorubicin was stronger in the MDA-MB-231 cancer cells. These results demonstrate that the tumor-targeting peptides increased specific drug delivery into cancer cells.

### Example 5: Efficacy Study of Different Ligand-MINC-Doxorubicin Against Cancer Cells

### Materials

RGD-MINC-doxorubicin, TfR-MINC-doxorubicin (TP12-MINC-doxorubicin) or MINC-doxorubicin was formulated according to Example 3.

Acid Phosphatase (ACP) Assay kit was purchase from LSBio.

MDA-MB-231 cell line was purchased from ATCC (HTB26).

### Method

Breast cancer cells MDA-MB-231 were seeded at 1×10⁴ cells/well in 96 well plate and incubated overnight. On the second day, cells were treated with MINC-doxorubicin, RGD-MINC-doxorubicin or TfR-MINC-doxorubicin at different concentrations for 72 hours. After the treatment, the cell viability was assessed by ACP kit following manufacturer's instruction. Optical density (OD) value was measured by spectrophotometer. Acid phosphatase is an enzyme present in cancer cells. The enzyme activity is positively correlated with cell viability and can be measured by OD value. The cell viability (%) is calculated by [(OD value of treated group−blank)/(OD value of untreated group−blank)]×100%.

### Result

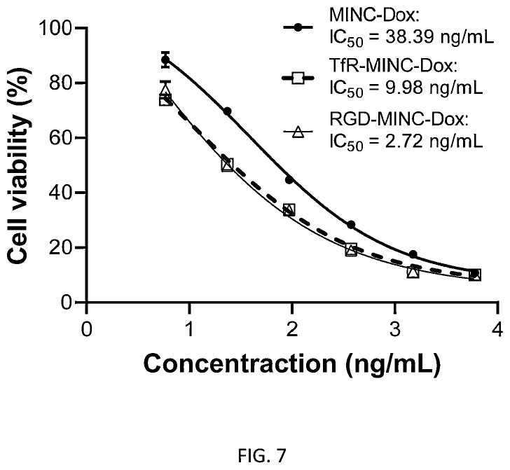

In FIG. 7, compared to bare MINC-doxorubicin, RGD-MINC-doxorubicin and TfR-MINC-doxorubicin treatment groups had less cell viability at the same concentration of doxorubicin. The calculated 50% inhibition concentration (IC50) were much lower in the RGD-MINC-doxorubicin and TfR-MINC-doxorubicin treatment groups than MINC-doxorubicin treatment group. The results demonstrate that the tumor targeting ligands improved the anti-cancer efficacy of MINC-doxorubicin.

### Example 6: RGD Peptide Conjugation to HO-PEG-EGCG (Prophetic Example)

### Objectives

This experiment is intended to demonstrate the conjugation of RGD to HO-PEG-EGCG. HPLC is used to detect the formation of new product (RGD-PEG-EGCG) with different retention time from HO-PEG-EGCG. NMR can be used to confirm the structure.

### Materials

RGD peptide is purchased from BIOTOOLS.

HO-PEG-CHO is purchased from Huanteng pharma.

### Method

HO-PEG-CHO is conjugated to EGCG according to WO2006/124000 and WO2009/054813 to prepare HO-PEG-EGCG.

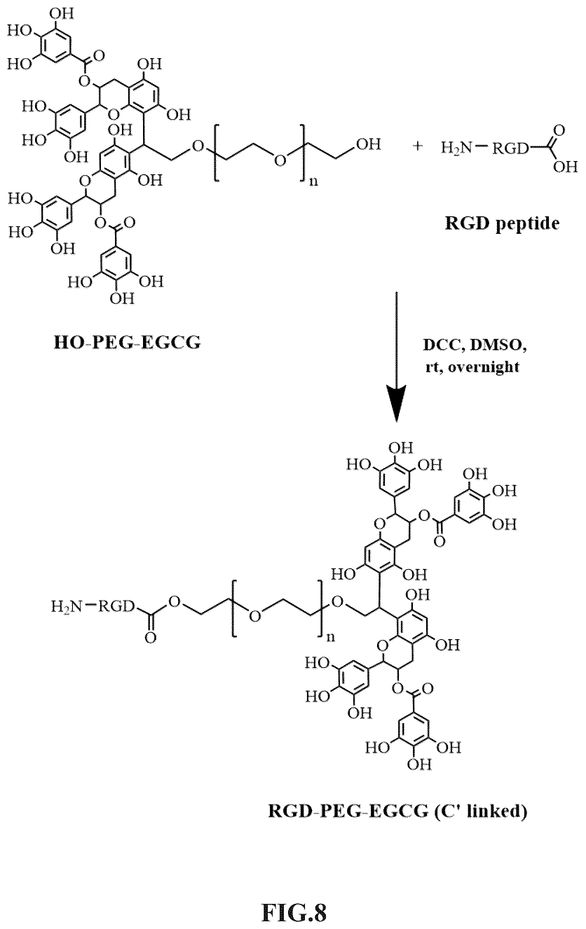

RGD is conjugated to HO-PEG-EGCG via the conjugation between OH group on PEG and COOH group on RGD to form RGD-PEG-EGCG (C′ linked). See FIG. 8.

Specifically, 1-1000 mg RGD is PEGylated by incubation with 1-1000 mg HO-PEG-EGCG, and 1-1000 mg N, N′-dicyclohexylcarbodiimide (DCC) in DMSO. The reaction is stirred at room temperature, prevented from light, under nitrogen for 24 hours. The reaction mixture is dialyzed (membrane Mw cutoff=2000 Da) against methanol and distilled water for 3 days. Next, the solution is freeze-dried to obtain lyophilized powder.

### Example 7: Transferrin Peptide (TP12) Conjugation to HO-PEG-EGCG (Prophetic Example)

### Objectives

This experiment is intended to demonstrate the conjugation of TP12 to HO-PEG-EGCG. HPLC is used to detect the formation of new product (TP12-PEG-EGCG) with different retention time from that of HO-PEG-EGCG. NMR can be used to confirm the structure.

### Materials

TP12 peptide is purchased from BIOTOOLS.

HO-PEG-CHO is purchased from Huanteng pharma.

### Method

For TP12-PEG-EGCG formulation, HO-PEG-CHO is conjugated to EGCG according to WO2006/124000 and WO2009/054813.

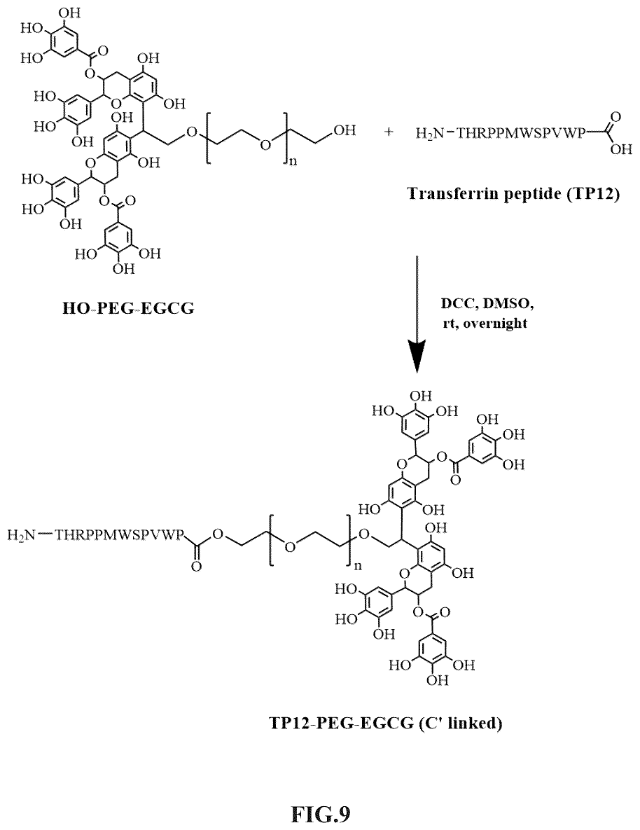

TP12 peptide is conjugated to HO-PEG-EGCG via the conjugation between OH group on PEG and COOH group on TP12 to form TP12-PEG-EGCG (C′ linked). See FIG. 9.

Specifically, 1-1000 mg TP12 is PEGylated by incubation with 1-1000 mg HO-PEG-EGCG, and 1-1000 mg N, N′-dicyclohexylcarbodiimide (DCC) in DMSO. The reaction is stirred at room temperature, prevented from light, under nitrogen for 24 hours. The reaction mixture is dialyzed (membrane Mw cutoff=2000 Da) against methanol and distilled water for 3 days. Next, the solution is freeze-dried to obtain lyophilized powder.

### Example 8: Folate Conjugation to HO-PEG-EGCG (Prophetic Example)

### Objectives

This experiment is intended to demonstrate conjugation of folate to HO-PEG-EGCG. HPLC is used to detect the formation of new product (Folate-PEG-EGCG) with different retention time from HO-PEG-EGCG. NMR can be used to confirm the structure.

### Materials

Folate is purchased from TCI chemicals.

HO-PEG-CHO is purchased from Huanteng pharma.

### Method

HO-PEG-CHO is conjugated to EGCG according to WO2006/124000 and WO2009/054813.

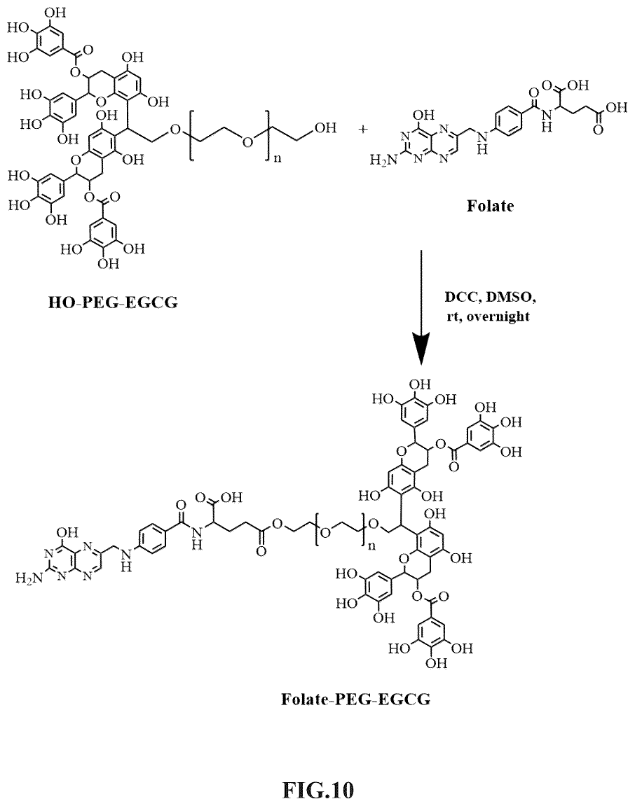

Folic acid or folate is conjugated to HO-PEG-EGCG via the conjugation between OH group on PEG and COOH or COOR group on folate to form folate-PEG-EGCG. See FIG. 10

Specifically, 1-1000 mg folate is PEGylated by incubation with 1-1000 mg HO-PEG-EGCG, and 1-1000 mg N, N′-dicyclohexylcarbodiimide (DCC) in DMSO. The reaction is stirred at room temperature, prevented from light, under nitrogen for 24 hours. The reaction mixture is dialyzed (membrane Mw cutoff=2000 Da) against methanol and distilled water for 3 days. Next, the solution is freeze-dried to obtain lyophilized powder.

### Example 9: RGD Peptide Conjugation to HOOC-PLA-EGCG (Prophetic Example)

### Objectives

This experiment is intended to demonstrate conjugation of RGD peptide to HOOC-PLA-EGCG. HPLC is used to detect the formation of new product (RGD-PLA-EGCG) with different retention time from HOOC-PLA-EGCG. NMR can be used to confirm the structure.

### Materials

RGD peptide is purchased from BIOTOOLS.

HOOC-PLA-CHO is purchased from Merck (Sigma-Aldrich).

### Method

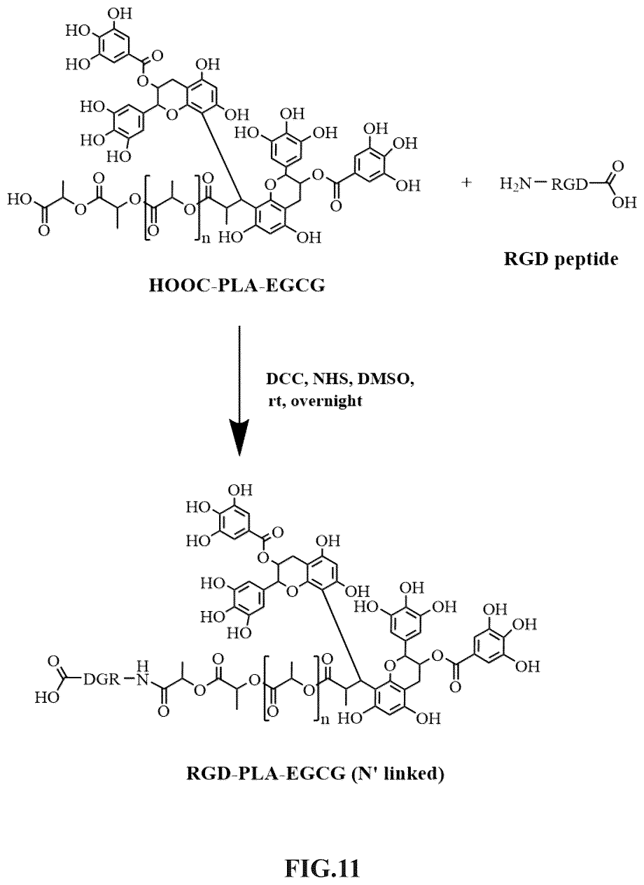

HOOC-PLA-CHO is conjugated to EGCG according to WO2006/124000 and WO2009/054813; RGD peptide is conjugated to HOOC-PLA-EGCG via the conjugation between COOH group on PLA and NH₂ group on RGD to form RGD-PLA-EGCG (N′ linked). See FIG. 11.

Specifically, 1-1000 mg RGD is incubated with 1-1000 mg HOOC-PLA-EGCG, 1-1000 mg N, N′-dicyclohexylcarbodiimide (DCC), and 1-1000 mg N-Hydroxysuccinimide (NHS) in DMSO. The reaction is stirred at room temperature, prevented from light, under nitrogen for 24 hours. The reaction mixture is dialyzed (membrane molecular weight cutoff=2000 Da) against methanol and distilled water for 3 days. Next, the solution is freeze-dried to obtain lyophilized powder.

### Example 10: RGD Peptide Conjugation to HOOC-PLGA-EGCG (Prophetic Example)

### Objectives

This experiment is intended to demonstrate conjugation of RGD peptide to HOOC-PLGA-EGCG. HPLC is used to detect the formation of new product (RGD-PLGA-EGCG) with different retention time from HOOC-PLGA-EGCG. NMR can be used to confirm the structure.

### Materials

RGD peptide is purchased from BIOTOOLS.

HOOC-PLGA-CHO is purchased from Merck (Sigma-Aldrich).

### Method

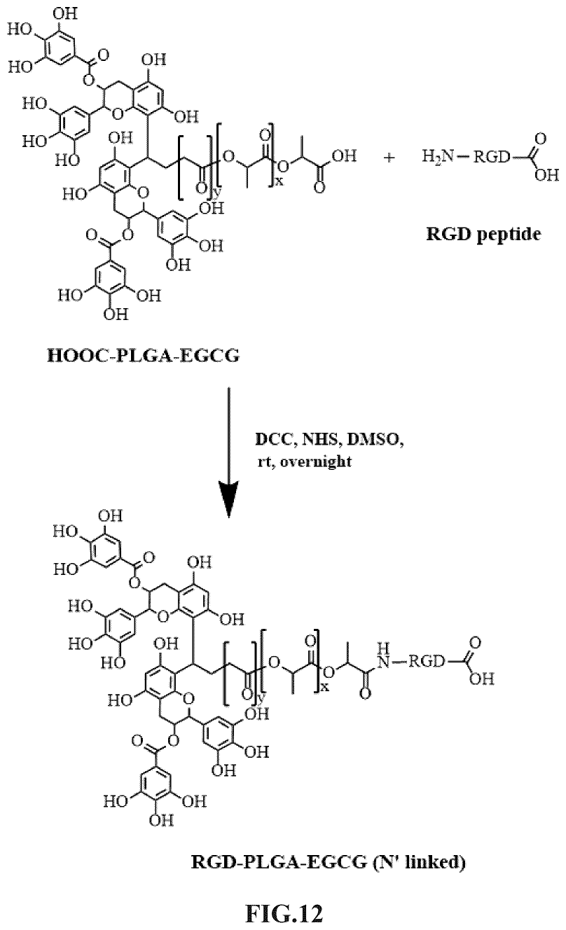

HOOC-PLGA-CHO is conjugated to EGCG according to WO2006/124000 and WO2009/054813; RGD peptide is conjugated to HOOC-PLGA-EGCG via the conjugation between COOH group on PLGA and NH₂ group on RGD to form RGD-PLGA-EGCG (N′ linked). See FIG. 12.

Specifically, 1-1000 mg RGD is incubated with 1-1000 mg HOOC-PLGA-EGCG, 1-1000 mg N, N′-dicyclohexylcarbodiimide (DCC), and 1-1000 mg N-Hydroxysuccinimide (NHS) in DMSO. The reaction is stirred at room temperature, prevented from light, under nitrogen for 24 hours. The reaction mixture is dialyzed (membrane molecular weight cutoff=2000 Da) against methanol and distilled water for 3 days. Next, the solution is freeze-dried to obtain lyophilized powder.

### Example 11: RGD Peptide Conjugation to HO-Dextran-EGCG (Prophetic Example)

### Objectives

This experiment is intended to demonstrate the conjugation of RGD to HO-Dextran-EGCG. HPLC is used to detect the formation of new product (RGD-Dextran-EGCG) with different retention time from HO-Dextran-EGCG. NMR can be used to confirm the structure.

### Materials

RGD peptide is purchased from BIOTOOLS.

HO-Dextran-CHO is purchased from Merck (Sigma-Aldrich).

### Method

HO-Dextran-CHO is conjugated to EGCG according to WO2006/124000 and WO2009/054813 to prepare HO-Dextran-EGCG.

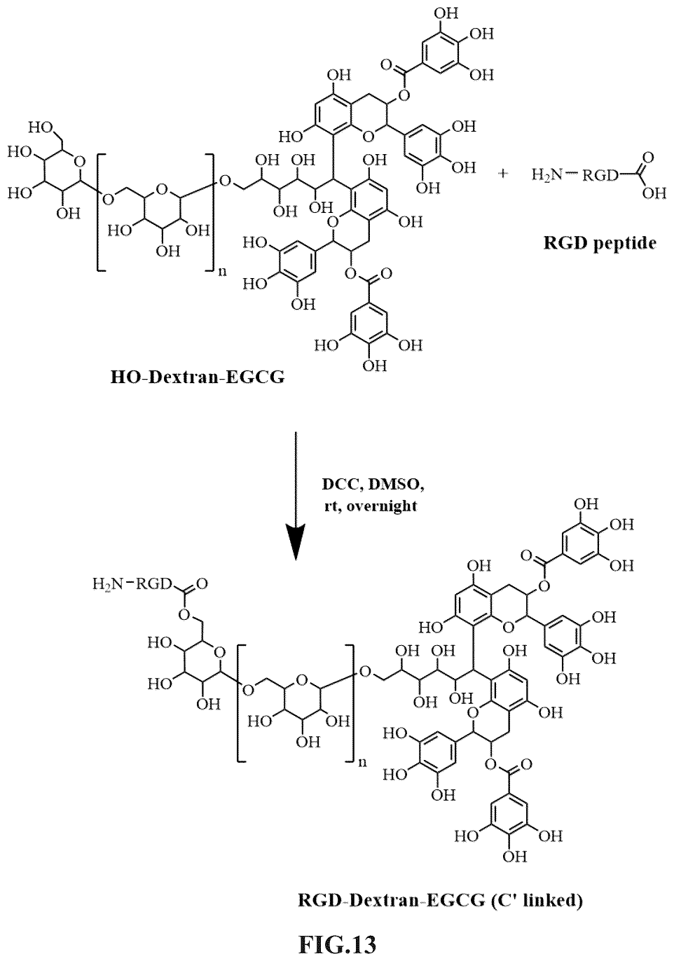

RGD is conjugated to HO-Dextran-EGCG via the conjugation between OH group on PEG and COOH group on Dextran to form RGD-Dextran-EGCG (C′ linked). See FIG. 13.

Specifically, 1-1000 mg RGD is incubated with 1-1000 mg HO-Dextran-EGCG, and 1-1000 mg N, N′-dicyclohexylcarbodiimide (DCC) in DMSO. The reaction is stirred at room temperature, prevented from light, under nitrogen for 24 hours. The reaction mixture is dialyzed (membrane Mw cutoff=2000 Da) against methanol and distilled water for 3 days. Next, the solution is freeze-dried to obtain lyophilized powder.

### Example 12: Formulation of Folate-MINC-Doxorubicin (Prophetic Example)

### Objectives

This experiment is intended to demonstrate the formulation of folate-MINC-doxorubicin. DLS is used to measure the size of nanoparticle.

### Materials

Folate-PEG-EGCG is formulated according to Example 8.

Doxorubicin is purchased from Sigma-Aldrich or other suppliers.

### Method

Folate-MINC-doxorubicin Nanoparticles are prepared according to the following protocol:
1. Incubate 5-500 μg doxorubicin in 1 mL DMSO for 15 min to 1 hour.
2. Add 1-100 μg of OEGCG and 1-10,000 μg of folate-PEG-EGCG. Incubate the mixture at 25° C. for 3 hours.
3. Filter out the liquid with a 10K MWCO filter unit. Wash the filter 3 time with 0.9% NaCl
4. Lyophilize to dry powder.

### LIST OF ABBREVIATIONS

AP-1 Activator Protein-1
AR Androgen Receptor
ASGPR Asialoglycoprotein Receptor
CCL2 Chemokine (C—C motif) Ligand 2
CCR2 C—C Chemokine Receptor Type 2
CD20 Cluster of Differentiation 20
CD44 Cluster of Differentiation 44
c-MET Mesenchymal-Epithelial Transition Factor
DR4 Death Receptor 4
EGa1 Ectodomain of Glycoprotein A1
EGF Epidermal Growth Factor
EGFR Epidermal Growth Factor Receptor
EpCAM Epithelial Cell Adhesion Molecule
ER Estrogen Receptor
Fas Fas cell surface death receptor
IL-13 Interleukin-13
IL-4R Interleukin-4 Receptor
IR Insulin Receptor
LHRH Luteinizing Hormone-Releasing Hormone
LRP5 Low-Density Lipoprotein Receptor-Related Protein 5
MMP1 Matrix Metalloproteinase 1
MUC1 Mucin 1
NOTCH1 Neurogenic Locus Notch Homolog Protein 1
PDGF Platelet-Derived Growth Factor
PD-L1 Programmed Death-Ligand 1
PEG Polyethylene glycol
PIGF Placental Growth Factor
PLA Polylactic acid
PLGA Polylactic-co-glycolic acid
PRPTK7 Protein Tyrosine Phosphatase Receptor Type K7
TbFGF Truncated basic Fibroblast Growth Factor
TNFR1 Tumor Necrosis Factor Receptor 1
WNT1 Wingless-Related Integration Site 1

What is claimed is:

1. A conjugate comprising:
(a) a cancer-targeting ligand, (b) a hydrophilic polymer of polyethylene glycol (PEG), polylactic acid (PLA), polylactic-co-glycolic acid (PLGA), or dextran, and (c) a flavonoid of EGCG, EC, EGC, or ECG, as shown in the structures below: 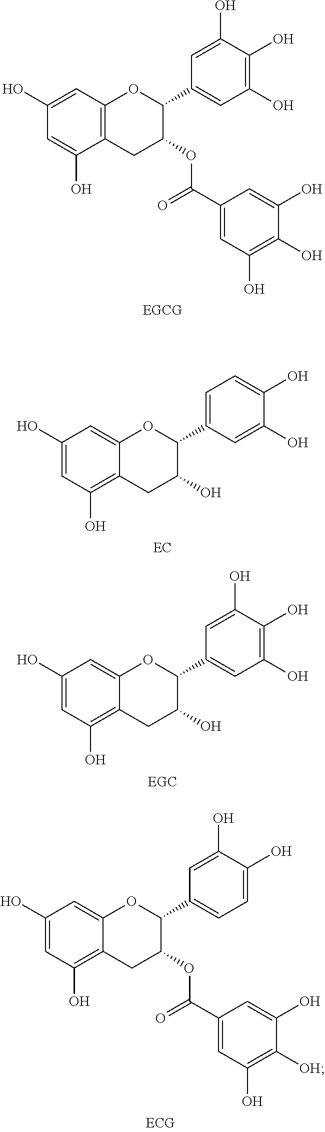 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]
wherein the hydrophilic polymer covalently binds to the flavonoid and the cancer-targeting ligand, wherein the cancer-targeting ligand is selected from the group consisting of:
RGD,
TP12 having the amino acid sequence of THRPPMWSPVWP (SEQ ID NO: 1),
MC11 having the amino acid sequence of MQLPLATGGGC (SEQ ID NO: 2),
FV12 having the amino acid sequence of FCDGFYACYMDV (SEQ ID NO: 3),
YI12 having the amino acid sequence of YHWYGYTPQNVI (SEQ ID NO: 4),
CTT having the amino acid sequence of CTTHWGFTLC (SEQ ID NO: 5),
H2009.1 peptide having the amino acid sequence of RGDLATLRQLAQEDGVVGVR (SEQ ID NO: 6),
IL-13 peptide having the amino acid sequence of GSETWKTIITKN (SEQ ID NO: 7),
AP-1 peptide having the amino acid sequence of RKRLDRN (SEQ ID NO: 8), CVKTPAQSC (SEQ ID NO: 9)
CC9 peptide having the amino acid sequence of CDCRGDCFC (SEQ ID NO: 10),
RGDS peptide having the amino acid sequence of RGDS (SEQ ID NO: 11),
NR7 peptide having the amino acid sequence of NSVRGSR (SEQ ID NO: 12),
LHRH peptide having the amino acid sequence of EHWSYGLRPG (SEQ ID NO: 13),
angiopep-2 peptide having the amino acid sequence of TFFYGGSRGKRNNFKTEEY (SEQ ID NO: 14),
TbFGF peptide having the amino acid sequence of KRTGQYKLC (SEQ ID NO: 15),
EGF peptide having the amino acid sequence of YHWYGYTPQNVI (SEQ ID NO: 16),
folic acid or folate, hyaluronic acid, lactose, galactose. galactosamine, and glycyrrhetinic acid.

2. The conjugate of claim 1, wherein the flavonoid is EGCG.

3. The conjugate of claim 1, wherein the hydrophilic polymer is PEG.

4. A nanoparticle composition comprising nanoparticles having: (a) an outer shell comprising the conjugate of claim 1, (b) an inner shell comprising oligomeric (−)-epigallocatechin gallate (OEGCG), and (c) a cancer-treating drug molecule of a cytokine, an antibody, or a chemotherapeutic agent encapsulated in the inner shell; wherein at least 70% of the nanoparticles have a diameter between 20-500 nm, and the size distribution of the nanoparticles only has one major peak containing more than 90% of all the particles.

5. The nanoparticle composition of claim 4, wherein at least 80% of the nanoparticles have a diameter between 50-300 nm.

6. The nanoparticle composition of claim 4, wherein at least 90% of the nanoparticles have a diameter between 50-300 nm.

7. The nanoparticle composition of claim 4, wherein the median diameter of the nanoparticles is about 50-250 nm.

8. The nanoparticle composition according to claim 4, wherein the size distribution of the nanoparticles has only one major peak containing more than 95% of all the particles.

9. The nanoparticle composition of claim 3, wherein the outer shell further comprises a bare hydrophilic polymer-flavonoid conjugate that does not covalently bind with the cancer-targeting ligand.

10. The nanoparticle composition of claim 9, wherein at least 80% of the nanoparticles have a diameter between 50-300 nm.

11. The nanoparticle composition of claim 9, wherein at least 90% of the nanoparticles have a diameter between 50-300 nm.

12. The nanoparticle composition of claim 9, wherein the median diameter of the nanoparticles is about 50-250 nm.

13. The nanoparticle composition according to claim 9, wherein the size distribution of the nanoparticles has only one major peak containing more than 95% of all the particles.

14. A method of treating cancer, comprising the step of administering to a subject in need thereof an effective amount of the conjugate of claim 1.

15. A method of treating cancer, comprising the step of administering to a subject in need thereof an effective amount of the nanoparticle composition of claim 4.
